(19) United States
(12) Patent Application Publication
(10) Pub. No.: US 2002/0119171 A1
(43) Pub. Date: Aug. 29, 2002

(54) COPOLYMERIC HYDROPHOBICALLY MODIFIED POLYASPARTIC ESTER AMIDES AND THEIR USE

(76) Inventors: BURGHARD GRUNING, ESSEN (DE); JORG SIMPELKAMP, ESSEN (DE); CHRISTIAN WEITEMEYER, ESSEN (DE)

Correspondence Address: LEOPOLD PRESSER, SCULLY SCOTT MURPHY & PRESSER, 400 GARDEN CITY PLAZA, GARDEN CITY, NY, 11530

(21) Appl. No.: 09/311,539

(22) Filed: May 13, 1999

(30) Foreign Application Priority Data
May 20, 1998 (DE) 198 22 599.7

(51) Int. Cl. A61K 31/74; A61K 9/00; A61K 6/00; A61K 7/00

(52) U.S. Cl. 424/401; 424/400; 424/78.01; 424/78.02; 424/78.03

(57) ABSTRACT

The present invention relates to copolymeric polyaspartic ester amides which have been modified with alkyl or alkenyl radicals having 6-30 carbon atoms, to their preparation and to their use.

Copolymers derived from polyamino acids, in which at least 75 mol % of the units present consist of structural units of the general formulae (I), (II) or (III)

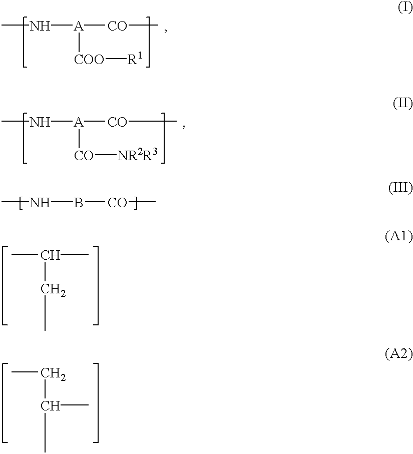

in which the structural elements A are identical or different trifunctional hydrocarbon radicals having 2 carbon atoms of the type (A1) or (A2), where

R¹ may have the meanings of R⁴, R⁵, and R⁶,

R² is identical or different, straight-chain or branched, saturated or unsaturated alkyl, alkenyl or aryl radicals having from 1 to 30 carbon atoms, hydroxyl or aminoalkyl radicals having from 1 to 22 carbon atoms and from 1 to 6 hydroxyl and/or amino groups and/or their acylation products with from 1 to 22 C carboxylic acids or assumes the meaning of R⁵, and

R³ is hydrogen or R²,

R⁴ is one or more radicals from the group consisting of alkali metals, alkaline earth metals, hydrogen or ammonium, [NR⁷R⁸R⁹R¹⁰]⁺, where R⁷ to R¹⁰ independently of one another are hydrogen, alkyl or alkenyl having from 1 to 22 carbon atoms or hydroxyalkyl having from 1 to 22 carbon atoms, having from 1 to 6 hydroxyl groups.

R⁵ is identical or different, straight-chain or branched, saturated or unsaturated alkyl or alkenyl radicals R¹¹ having from 6 to 30 carbon atoms or radicals of the structure —Y—R¹¹, where Y is an oligo- or polyoxyalkylene chain having from 1 to 100 oxyalkylene units, and

R⁶ is identical or different, straight-chain or branched, saturated or unsaturated alkyl or alkenyl radicals having from 1 to 5 carbon atoms, and in each case at least one unit of the general formula (I) in which the radical R¹ assumes the meaning of R⁴, and at least one unit of the general formula (II) is present, and the units of the general formula (III) are proteinogenic or nonproteinogenic amino acids and are present in amounts of no more than 20% by weight, based on copolymeric polyaspartic acid derivatives.

### BACKGROUND OF THE INVENTION

1. Technical Field

The present invention relates to copolymeric polyaspartic ester amides which have been modified with alkyl or alkenyl radicals having 6-30 carbon atoms, to their preparation and to their use.

2. Prior Art

Polyamino acid derivatives, in particular polyaspartic acid, have recently attracted particular attention because of their properties, in particular their biodegradability and their similarity to naturally occurring structures. Proposed applications are inter alia as biodegradable complexing agents, water softeners and detergent builders. Polyaspartic acid is generally obtained by alkaline hydrolysis of the direct synthesis precursor polysuccinimide (PSI, anhydropolyaspartic acid), the cyclic imide of polyaspartic acid. PSI can be prepared, for example, in accordance with EP 0 578 449 A, WO 92/14753, EP 0 659 875 A or DE 44 20 642 A from aspartic acid, or is obtainable, for example according to DE 36 26 672 A, EP 0 612 784 A, DE 43 00 020 A or U.S. Pat. No. 5,219,952 A, from maleic acid derivatives and ammonia. Proposed applications for these customary polyaspartic acids are inter alia as encrustation inhibitors, builders in detergents, fertilizer additive and additive in tanning.

The reaction of polysuccinimide with amines, which has been described by various working groups, leads to polyaspartic amides (Kovacs et al., J. Med. Chem. 1967, 10, 904-7; Neuse, Angew. Makromol. Chem. 1991, 192, 35-50). The ring opening of polysuccinimide using polyamines and the subsequent alkaline hydrolysis for the preparation of polyaspartic acid derivatives for applications as superabsorbers is described, for example, in WO 95/35337.

U.S. Pat. No. 5,292,858 A describes the reaction of maleic monoesters with ammonia or ammonia and amines to give polysuccinimide or amide-group-containing polysuccinimide derivatives which no longer contain ester groups in the final product.

For applications inter alia as emulsifier, dispersant and surfactant, copolymeric polyaspartic esters partially esterified with long-chain fatty alcohols or their derivatives are of particular interest. Such compounds are readily obtainable on the basis of maleic monoesters and ammonia, as explained in DE 195 45 678 A or EP 96 118 806.7 A.

In terms of their technical properties, however, these derivatives have various disadvantages, in particular as regards thermal and long-term stability of the preparations, for example in the field of cosmetic W/O and O/W emulsions.

The object of the invention is therefore to provide copolymeric polyaspartic ester derivatives which have improved application properties.

### SUMMARY OF THE INVENTION

The object is achieved according to the invention by copolymeric polyaspartic esters having ester and amide groups in the polymer side chain which are prepared from derivatives of α, β-unsaturated carboxylic acids and ammonia. The simultaneous presence of ester and amide groups in the molecule surprisingly gives particularly advantageous application properties.

### DETAILED DESCRIPTION OF THE INVENTION

The copolymers derived from polyaspartic acid which are used comprise, in amounts up to at least 75 mol % of the units present, structural units of the general formulae (I), (II) and (III), the structural elements A being identical or different trifunctional hydrocarbon radicals having 2 carbon atoms of the structure (A1) or (A2),

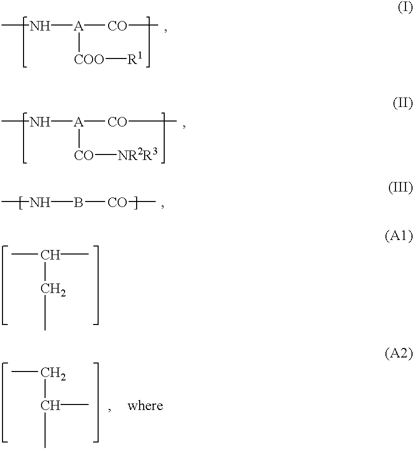

R¹ is as defined for R⁴, R⁵, and R⁶,

R² is identical or different, straight-chain or branched, saturated or unsaturated alkyl, alkenyl or aryl radicals having from 1 to 30 carbon atoms, hydroxyl or aminoalkyl radicals having from 1 to 22 carbon atoms and from 1 to 6 hydroxyl and/or amino groups and/or their acylation products with from 1 to 22 C carboxylic acids or assumes the meaning of R⁵, and

R³ is hydrogen or R²,

R⁴ is one or more radicals from the group consisting of alkali metals, alkaline earth metals, hydrogen or ammonium, [NR⁷R⁸R⁹R¹⁰]⁺, where R⁷ to R¹⁰ independently of one another are hydrogen, alkyl or alkenyl having from 1 to 22 carbon atoms or hydroxyalkyl having from 1 to 22 carbon atoms and from 1 to 6 hydroxyl groups.

R⁵ is identical or different, straight-chain or branched, saturated or unsaturated alkyl or alkenyl radicals R¹¹ having from 6 to 30 carbon atoms or radicals of the structure —Y—R¹¹, where Y is an oligo or polyoxyalkylene chain having from 1 to 100 oxyalkylene units, and

R⁶ is identical or different, straight-chain or branched, saturated or unsaturated alkyl or alkenyl radicals having from 1 to 5 carbon atoms, and at least in each case one unit (I) in which the radical R¹ assumes the meaning of R⁴, and at least one unit (I) in which the radical R¹ assumes that of R⁵ or R⁶, and at least one unit (II) is present, and the units of the general formula (III) are proteinogenic or nonproteinogenic amino acids and are present in amounts of no more than 20% by weight, based on copolymeric polyaspartic acid derivatives.

All data given relating to the composition of the polymeric products refer, as is usual, to the average composition of the polymer chains.

Preference is given to using products in which at least one radical R¹ or R² assumes the meaning of R⁵.

The remaining units (maximum 25 mol % which do not have the structure (I), (II) or (III)) can inter alia be iminodisuccinate units of the structure (IV)

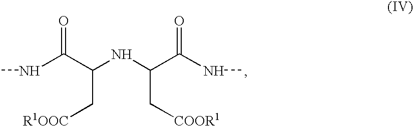

and various end groups, on the N terminus for example aspartic acid, maleic acid, fumaric acid and malic acid units and their esters or amides, maleimide units or diketopiperazines derived from aspartic acid and/or the amino acid units (III), and esters or amides of the amino acid units (III), on the C terminus for example aspartic or malic acid units, their mono- or diesters, amides or cyclic imides.

Suitable amino acid units (III) from the group of proteinogenic amino acids are, for example, glutamine, asparagine, lysine, alanine, glycine, tyrosine, tryptophan, serine and cysteine and their derivatives; nonproteinogenic amino acids can, for example, be (-alanine, (-amino-1-alkanoic acids etc.

The units (II) are preferably derived from primary or secondary amines NR²R³H, where R² is a straight-chain or branched, saturated, or unsaturated alkyl, alkenyl or aryl radical having from 1 to 30 carbon atoms, particularly preferably an alkyl radical having from 8 to 24 carbon atoms (for example branched or linear octyl, decyl, dodecyl, tetradecyl, hexadecyl, octadecyl, docosyl radicals, also unsaturated and polyunsaturated species such as, for example, oleyl), and R³ is hydrogen or methyl. Other preferred amines are aminoalcohols, for example ethanolamine, diethanolamine, aminopropanol, their N-methyl derivatives and their acylation products, for example with straight-chain or branched, saturated, or unsaturated carboxylic acids having from 1 to 30 carbon atoms, particularly preferably having from 8 to 24 carbon atoms.

A preferred form of the novel copolymers comprises at least one free carboxylate group (R¹═H, metal, ammonium), at least one unit (I) in which R¹ is as defined for R⁵ or R⁶, and at least one unit (II) which comprises, as radical R², an alkyl or alkenyl radical from the group of straight-chain or branched, saturated or unsaturated alkyl radicals having from 8 to 24 carbon atoms (e.g. branched or linear octyl, decyl, dodecyl, tetradecyl, hexadecyl, octadecyl, docosyl radicals, also unsaturated and polyunsaturated species such as, for example, oleyl).

A further preferred form of the novel copolymers comprises at least one free carboxylate group R¹═H, metal, ammonium), at least one unit (I) in which R¹ is as defined for R6, and R⁶ originates from the group of straight-chain or branched, saturated or unsaturated alkyl or alkenyl radicals having from 1 to 5 carbon atoms (e.g. methyl, ethyl, n-propyl, i-propyl, n-butyl, i-butyl, s-butyl, n-pentyl), and at least one unit (II) which comprises, as radical R², an alkyl or alkenyl radical from the group of straight-chain or branched, saturated or unsaturated alkyl or alkenyl radicals having from 8 to 24 carbon atoms (for example branched or linear octyl, decyl, dodecyl, tetradecyl, hexadecyl, octadecyl, docosyl radicals, also unsaturated and polyunsaturated species such as, for example, oleyl). Particular preference is given to compounds in which R³ is hydrogen or methyl.

A further preferred form of the novel copolymers comprises at least one free carboxylate group (R¹═H, metal, ammonium), at least one unit (I) in which R¹ is as defined for R⁵, and R⁵ is identical or different, straight-chain or branched, saturated or unsaturated alkyl or alkenyl radicals R¹¹ having from 6 to 30 carbon atoms or radicals of the structure —Y—R¹¹, where Y is an oligo- or polyoxyalkylene chain having from 1 to 100 oxyalkylene units, and at least one unit (II) which comprises, as radical R², an alkyl or alkenyl radical from the group of straight-chain or branched, saturated or unsaturated alkyl or alkenyl radicals having from 1 to 30 carbon atoms, particularly preferably from 1 to 8 carbon atoms. Particular preference is given to compounds having alkyl or alkenyl radicals R⁵ without alkylene glycol spacers and having radicals R³ which are hydrogen or methyl.

The novel copolymers can be obtained, for example, by reacting a mixture of monoesters and monoamides, optionally in the presence of diesters and/or diamides and/or anhydrides, of monoethylenically unsaturated dicarboxylic acids with 0.5-1.5 equivalents of ammonia, or by thermally converting the ammonium salts of these acids into the polymer. It is possible to use, for example, derivatives of maleic acid, fumaric acid, itaconic acid, alkenylsuccinic acid, alkylmaleic acid, citraconic acid or their ammonium salts, preferably derivatives of maleic acid, fumaric acid or itaconic acid, particularly preferably maleic acid derivatives of the general formulae (V), (VI) and (VII)

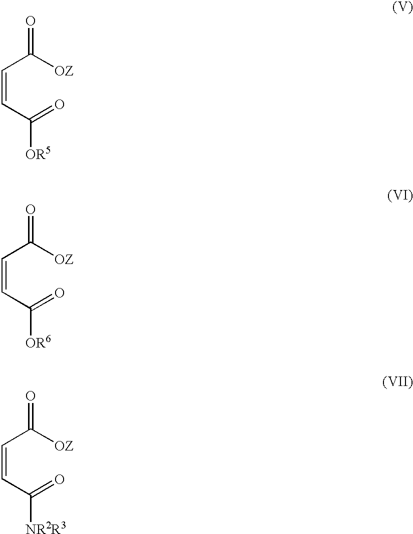

where Z is hydrogen or ammonium, R², R³, R⁵ and R⁶ are the abovementioned radicals, and the amount of (VII) is not 0, nor is the total of the amounts of (V) and (VI). Use is made of mixtures having a total content of from 0.5 to 99% by weight, preferably from 10 to 90% by weight, particularly preferably from 40 to 95% by weight, of ester component (V) and/or (VI) and having from 1 to 100% by weight, preferably from 10 to 90% by weight, particularly preferably from 5 to 60% by weight, of amide component (VII).

Preferably used radicals R⁵ are alkyl or alkenyl radicals having from 8 to 30 carbon atoms, for example linear or branched decyl, dodecyl, tetradecyl, hexadecyl, or octadecyl radicals and unsaturated alkyl or alkenyl radicals, such as, for example, oleyl. Preferably used radicals R⁴ are alkyl radicals having from 1 to 4 carbon atoms, preferably methyl, ethyl, n-propyl, i-propyl, n-butyl, i-butyl, s-butyl.

The reaction can be carried out with or without the addition of organic solvents. Examples of suitable solvents are alcohols, ketones, esters, oligo- and poly(alkylene) glycols and glycol ethers, dimethyl sulfoxide, dimethylformamide, N,N-dimethylacetamide or N-methylpyrrolidone and their mixtures, etc. Preference is given to using alcohols having 2-4 carbon atoms, and of these particularly preferably the short-chain alcohol R⁴OH, and ketones such as, for example, methyl isobutyl ketone or methyl isoamyl ketone, or alkyl esters of carboxylic acids having 1-4 carbon atoms, such as, for example, sec-butyl acetate or pentyl acetate.

The reaction can optionally be carried out in the presence of compatibility-promoting agents. These may be surface-active compounds, for example addition products of from 1 to 30 mol of ethylene oxide and/or from 0 to 5 mol of propylene oxide with C₁₂-C₃₀-fatty alcohols and wool wax alcohols; ethylene oxide addition products of glycerol mono- and diesters and sorbitan mono- and diesters of saturated and unsaturated fatty acids having from 6 to 22 carbon atoms; addition products of from 2 to 30 mol of ethylene oxide and/or from 0 to 5 mol of propylene oxide with fatty acids having from 12 to 22 carbon atoms and with alkylphenols having from 8 to 15 atoms in the alkyl group, C₁₂-C₁₈-fatty acid partial esters of addition products of from 1 to 30 mol of ethylene oxide with glycerol; addition products of ethylene oxide with fats and oils, for example castor oil or hydrogenated castor oil; partial esters of saturated or unsaturated C₁₂-C₂₂-fatty acids, including branched or hydroxyl-substituted ones, with polyols, for example esters of glycerol, ethylene glycol, polyalkylene glycols, pentaerythritol, polyglycerol, sugar alcohols such as sorbitol, and polyglucosides such as cellulose; polysiloxane-polyalkyl-polyether copolymers and their derivatives and hydrophobically modified polyaspartic acid derivatives, for example partially esterified polyaspartic acids, partially esterified polyaspartic acid-co-glutamic acids or condensates of maleic monoesters and ammonia, prepared, for example, by the novel process or as in DE 195 45 678 A, the preparation process of said polyamino acid derivatives having no influence on their compatibility-promoting effect. If desired, a certain portion of the product mixture may also remain in the reactor and be used as solubilizer for a subsequent reaction.

The compatibility- or solubility-promoting agents present may also be cationic surfactants, for example from the group of quaternary ammonium compounds, quaternized protein hydrolysates, alkylamido amines, quaternary ester compounds, quaternary silicone oils, quaternary sugar and polysaccharide derivatives, anionic surfactants, for example from the group of sulfates, sulfonates, carboxylates and mixtures thereof, for example alkylbenzenesulfonates, (-olefinsulfonates, (-sulfonated fatty acid esters, fatty acid glycerol ester sulfates, paraffinsulfonates, alkyl sulfates, alkyl polyether sulfates, alkyl sulfosuccinates, fatty acid salts (soaps), fatty acid esters of polylactic acid, N-acylamino acid esters, N-acyltaurates, acylisethionates, ethercarboxylates, monoalkyl phosphates, N-acylamino acid derivatives, such as N-acylaspartates or N-acylglutamates, N-acyl-sarcosinates, amphoteric or zwitterionic surfactants, such as, for example, alkylbetaines, alkylamidoalkylbetaines of the cocoamidopropylbetaine type, sulfobetaines, phosphobetaines, sultaines and amidosultaines, imidazolinium derivatives, amphoglycinates, or nonionic surfactants, such as, for example, oxethylated fatty alcohols, oxethylated alkylphenols, oxethylated fatty acid esters, oxethylated mono-, di- or triglycerides or polyalkylene glycol fatty acid esters, sugar esters, for example fatty acid esters of sucrose, fructose or of methyl glucoside, sorbitol fatty acid esters and sorbitan fatty acid esters (optionally oxethylated), alkyl or alkenyl polyglucosides and their ethoxylates, fatty acid N-alkylpolyhydroxyalkylamides, polyglycerol esters, fatty acid alkanolamides, long-chain tertiary amine oxides or phosphine oxides, and dialkyl sulfoxides.

The compatibility-promoting agents preferably remain in the product. The reaction to give the copolymers is carried out in a preferred procedure with aqueous or gaseous ammonia at temperatures of from 0 to 150° C., preferably 50-140° C., and subsequent distillation is carried out at from 70 to 240° C., preferably from 110 to 150° C., under reduced pressure, for example in kneading devices, high-viscosity reactors, extruders and stirred reactors, optionally using high-shear force stirrers such as Mig or Intermig stirrers.

Under the reaction conditions, some of the ester groups, preferably those derived from R⁶OH, are also hydrolyzed and the desired carboxylic acid or carboxylate groups liberated. Subsequent mild partial or complete hydrolysis, preferably of the ester functions derived from the short-chain alcohol R⁶OH, can, if desired, increase further the amount of free acid groups, for example by reaction with water, optionally in the presence of acids or bases, or with alkali metal hydroxides, optionally in the presence of an organic solvent or cosolvent.

The ester and amide components can in each case be any mixtures of compounds having different radicals R², R³, R⁵, and R⁶.

Instead of maleic monoesters and -amides, it is also possible to use directly maleic anhydride mixed with the corresponding alcohols and amines.

By adding amino- and carboxy-functional compounds to the reaction mixture, it is possible to obtain copolymers in which the offered units are bonded via amide bonds. Suitable units are amino acids from the group of the 20 proteinogenic amino acids which are present as monomers in all natural proteins, in an enantiomerically pure or racemic form, such as, for example, glutamic acid, glutamine, asparagine, lysine, alanine, glycine, tyrosine, tryptophan, serine and cysteine and their derivatives, or nonproteinogenic amino acids having in each case one or more amino or carboxy functions, such as, for example β-alanine, ω-amino-l-alkanoic acids, for example 6-aminocaproic acid. The units, preferably from 0 to 15% by weight, are added to the starting mixture of the maleic acid derivatives or, for modification of the chain ends, are reacted therewith after synthesis of the polymer has taken place, preferably with the addition of polar solvents, such as, for example, alcohols or dimethylformamide.

The molecular mass of the polyaspartic acid derivatives can be increased by adding di- and/or polyfunctional units derived from a di- or polyhydroxy compound, a di- or polyamino compound, or amino alcohols having a linear, branched or cyclic, saturated, unsaturated or aromatic hydrocarbon backbone, optionally oxo or aza analogs having O or N atoms in the chain, or from polyalkylene glycols or ethylene oxide/propylene oxide copolymers. The groups which increase the molecular mass are introduced by adding the polyfunctional amino or hydroxyl compounds or their reaction products with maleic anhydride to the reaction mixture or to the polymer formed, optionally with the addition of acidic or Lewis-acidic catalysts.

The procedures described can also be combined.

The resulting polymers can be post-treated, for example by treatment with ammonia, transesterification catalysts such as, for example, Lewis-acid titanium(Iv) compounds, with activated carbon or other adsorbents, bleaching with oxidizing agents such as H₂O₂, Cl₂, O₃, sodium chlorite, sodium hypochlorite etc. or reducing agents such as, for example, NaBH₄ or H₂ in the presence of catalysts, under customary conditions.

The novel copolymers have excellent properties as sequestering agents, as additives for colorants and coatings, as foam stabilizers, surfactants and emulsifiers. In particular, the thermal and long-term stability of O/W and W/O emulsions is beneficially influenced.

The novel polymers can be used as O/W emulsifiers for cosmetic emulsions, for example for lotions having a comparatively low viscosity or creams and ointments having a high viscosity, for applications as skin care compositions, such as, for example, day creams, night creams, care creams, nourishing creams, body lotions, ointments and the like. Other auxiliaries and additives which may be present are customary coemulsifiers, bodying agents, oily substances, superfatting agents, fats, waxes, stabilizers, active ingredients, glycerol, dyes and fragrances.

Suitable bodying agents which may be used are hydrophilic waxes, for example C₁₂-C₃₀ fatty alcohols, C₁₆-C₂₂ fatty acids, glycerol mono- and diesters and sorbitan mono- and diesters of saturated fatty acids having from 12 to 22 carbon atoms.

Examples of other suitable coemulsifiers are: addition products of from 2 to 30 mol of ethylene oxide and/or from 0 to 5 mol of propylene oxide with C₁₂-C₃₀ fatty alcohols and wool wax alcohols, preferably linear, saturated C₁₆-C₂₂ fatty alcohols; ethylene oxide addition products of glycerol mono- and diesters and sorbitan mono- and diesters of saturated and unsaturated fatty acids having from 6 to 22 carbon atoms; addition products of from 2 to 30 mol of ethylene oxide and/or from 0 to 5 mol of propylene oxide with fatty acids having from 12 to 22 carbon atoms and with alkyl-phenols having from 8 to 15 carbon atoms in the alkyl group; C₁₂-C₁₈fatty acid partial esters of addition products of from 1 to 30 mol of ethylene oxide with glycerol; addition products of ethylene oxide with fats and oils, for example castor oil or hydrogenated castor oil; partial esters of saturated or unsaturated C₁₂-C₂₂ fatty acids, including branched or hydroxy-substituted ones, with polyols, for example esters of glycerol, ethylene glycol, polyalkylene glycols, pentaerythritol, polyglycerol, sugar alcohols such as sorbitol and polyglucosides such as cellulose; polysiloxane-polyalkyl-polyether copolymers and their derivatives and hydrophobically modified polyaspartic acid derivatives.

The selected coemulsifiers may also be anionic, cationic, nonionic, amphoteric and/or zwitterionic surfactants, for example from the group referred to as compatibility-promoting agents.

It is possible in each case to use any mixtures of the above bodying agents and coemulsifiers.

Examples of suitable oily substances are esters of linear C₆-C₂₀ fatty acids with linear C₆-C₂₀ fatty alcohols, esters of branched C₆-C₁₃ carboxylic acids with linear C₆-C₂₀ fatty alcohols, esters of linear C₆-C₂₀ fatty acids with branched alcohols, esters of linear and/or branched C₆-C₂₀ carboxylic acids with polyhydric alcohols and/or Guerbet alcohols, triglycerides based on C₆-C₁₀ fatty acids, vegetable and animal oils and fats, branched primary alcohols, substituted cyclohexanes, Guerbet carbonates, dialkyl ethers and/or aliphatic or naphthenic hydrocarbons.

Suitable superfatting agents are, for example, lanolin and lecithin derivatives and their ethoxylates, polyol fatty acid esters, monoglycerides and fatty acid alkanolamides. Silicone compounds such as polydimethylsiloxanes, cyclodimethicones and amino-, fatty-acid-, alcohol-, epoxy-, fluorine-, and/or alkyl-modified silicone compounds, and waxes such as, for example, beeswax, paraffin waxes or microcrystalline waxes may be present. The emulsions may comprise thickeners, such as polyacrylic acid derivatives or cationic polymers such as, for example, cationic cellulose or starch derivatives, cationic chitin or chitosan derivatives, cationic silicone polymers, copolymers of diallylammonium salts e.g. with acrylamides, polyethylenimine. Furthermore, metal salts of fatty acids, e.g. magnesium, aluminum or zinc stearate, may be present as stabilizers, or zinc salts of ricinoleic acid may be present as deodorizers. Customary sunscreen active ingredients such as titanium dioxide, p-aminobenzoic acid etc., fragrances, dyes, biogenic active ingredients such as plant extracts or vitamin complexes and pharmaceutical active ingredients may be present. The emulsions may further comprise lusterizing agents, such as ethylene glycol distearate and customary preservatives such as parabens, sorbic acid, phenoxyethanol and others.

The novel polyaspartic acid derivatives can also be used in W/O emulsions, for example as emulsifiers and/or coemulsifiers for the preparation of skincare creams and lotions.

The novel polyaspartic acid derivatives having a polyamino acid backbone which is similar to naturally occurring structures are mild surfactants which can be used alone or in combination with anionic, cationic, nonionic, zwitterionic and/or amphoteric surfactants. Solid, liquid or paste preparations are possible, e.g. soap bars, washing lotions, shower gels, shampoos.

The surfactants which can be used in combination with the novel polyaspartic acid derivatives can for example be anionic surfactants from the group of sulfates, sulfonates, carboxylates and mixtures thereof. The anionic groups can be in neutralized form, containing cationic counterions from the group of alkali metals, alkaline earth metals, ammonium or substituted ammonium. Use can be made, for example, of alkylbenzenesulfonates, a-olefinsulfonates, a-sulfonated fatty acid esters, fatty acid glycerol ester sulfates, paraffinsulfonates, alkyl sulfates, alkyl polyether sulfates, alkyl sulfosuccinates, fatty acid salts (soaps), fatty acid esters of polylactic acid, N-acylamino esters, N-acyltaurates, acyl-isethionates, ether carboxylates, monoalkyl phosphates, N-acylamino acid derivatives, such as N-acylaspartates or N-acylglutamates, N-acylsarcosinates, polyaspartic acid derivatives and others.

The surfactants which can be used in combination with the novel polyaspartic acid derivatives can, for example, be amphoteric or zwitterionic surfactants, for example alkylbetaines, alkylamidoalkylbetaines of the cocoamidopropylbetaine type, sulfobetaines, phosphobetaines, sultaines and amidosultaines, imidazolinium derivates, amphoglycinates and others.

The cationic surfactants which can be used in combination with the novel polyaspartic acid derivatives can, for example, be from the group of quaternary ammonium compounds, quaternized protein hydrolysates, alkylamido amines, quaternary ester compounds, quaternary silicone oils or quaternary sugar and polysaccharide derivatives.

The surfactants which can be used in combination with the novel polyaspartic acid derivatives can, for example, be nonionic surfactants, for example oxethylated fatty alcohols, oxethylated alkylphenols, oxethylated fatty acid esters, oxethylated mono-, di- or triglycerides or polyalkylene glycol fatty acid esters. Other nonionic surfactants can originate from the group of alkyl polysaccharides, for example alkyl or alkenyl polyglucosides, sugar esters, for example fatty acid esters of glucose, sucrose, fructose or of methyl glucoside, sorbitol fatty acid esters and sorbitan fatty acid esters (optionally oxethylated), polyglycerol esters, fatty acid alkanolamides, N-acylamino sugar derivatives, for example N-acylglucamines, long-chain tertiary amine oxides or phosphine oxides, and dialkyl sulfoxides.

The surfactants which can be used in combination with the novel polyaspartic acid derivatives can thus also be any combinations of two or more surfactants from the above categories.

The surfactant preparations according to the invention can comprise further auxiliaries and additives, such as, for example, water and solvents, for example from the group of alcohols and polyols, thickeners, opacifiers, e.g. glycol ester derivatives; moisturizers, emollients such as animal and vegetable oils, carboxylic esters, lanolin, beeswax, silicones; polymeric agents for improving the feel on the skin, conditioning, care or pharmaceutically active constituents such as, for example, cationic or amphoteric polymers, proteins and protein derivatives, lanolin derivatives, pantothenic acid, betaine, polydimethylsiloxanes or their derivatives, sunscreen active ingredients and solubilizers, stabilizers, fragrances, buffer substances, preservatives and/or dyes.

The surfactant preparations comprising polyaspartic acid derivatives can advantageously be used in, for example, hair shampoos, shower preparations, bubble bath preparations, hand, face and intimate area cleansing lotions, liquid soaps, soap bars, shaving creams, handwashing pastes, dishwashing detergents which are gentle on the skin, cleaners for smooth surfaces and in toothpastes.

The novel polyaspartic acid derivatives can be used as dispersants, for example for coatings and colorants.

The novel hydrophobically modified polyaspartic acid derivatives are, if they are not already present as a salt, neutralized advantageously using prior art neutralizing agents, in particular amines. Particular preference is here given to using dimethylethanolamine or 2-amino-2-methylpropanol. For the preparation of aqueous pigment pastes, 0.1-100% by weight, preferably 0.5-50% by weight, in particular 2 to 15% by weight, based on the weight of the pigments, are used. The hydrophobically modified polyaspartic acid derivatives can, for the novel use, either be mixed beforehand with the pigments to be dispersed, or be dissolved directly in the dispersing medium (water, possible additions of glycol) prior to or at the same time as the addition of the pigments and any other solids. Neutralization can take place before or during preparation of the pigment pastes. Preference is given to using polyaspartic acid preparations which have already been partially or completely neutralized.

The novel polyaspartic acid derivatives can also be used in any mixtures with other, prior art dispersion additives, for example from the group of fatty acid alkoxylates, poly(meth)acrylates, polyesters, polyethers etc.

In this connection, examples of suitable pigments are inorganic or organic pigments, and carbon black. Examples of inorganic pigments are titanium dioxide and iron oxides. Suitable organic pigments are, for example, azo pigments, metal complex pigments, phthalocyanine pigments, anthraquinoid pigments, polycyclic pigments, in particular those from the thioindigo, quinacridone, dioxazine, pyrrolopyrrole, naphthalenetetracarboxylic acid, perylene, isoamidolin(on)e, flavanthrone, pyranthrone or isoviolanthrone series.

Fillers which can, for example, be dispersed in aqueous coatings are, for example, those based on kaolin, talc, other silicates, chalk, glassfibers, glass beads or metal powders.

Suitable coating systems into which the novel pigment pastes can be incorporated are any aqueous 1-component or 2-component coatings. Examples include aqueous 1-component coatings, such as, for example, those based on alkyd, acrylate, epoxy, polyvinyl acetate, polyester or polyurethane resins, or aqueous 2-component coatings, for example those based on hydroxyl-group-containing polyacrylate or polyester resins with melamine resins or optionally blocked polyisocyanate resins as crosslinkers. Polyepoxy resin systems may likewise also be mentioned.

The novel polyaspartic acid derivatives can be used as complexing agents, for example in detergents, as encrustation inhibitors, as metal deactivators in plastics, as auxiliaries in the paper, leather and textile industries or as activity-enhancing additives for pesticides or insecticides. High molecular weight derivatives, preferably after modification with the abovementioned polyfunctional hydroxy and amino compounds, are also suitable as absorber materials.

### EXAMPLES

### Examples 1 to 6

The starting materials (monoethyl maleate, monoalkyl maleate, N-alkylmaleamide according to Tab. 1, dissolved in 4-methyl-2-pentanone) were reacted with from 1.0 to 1.5 equivalents of ammonia gas, and the reaction mixture was distilled at from 110° C. to 140° C. under reduced pressure for 5 h.

TABLE 1
Synthesis of polyaspartic acid derivatives

| | | | | | | | | PRODUCT: | PRODUCT: | PRODUCT: | PRO-DUCT: |
|---|---|---|---|---|---|---|---|---|---|---|---|
| | | MOL OF | | MOL OF | | | MOL OF | MOL % OF | MOL % OF | MOL % OF | MOL % OF |
| EXAMPLE | R⁵ | (v) | R⁶ | (VI) | R² | R³ | (VII) | COOR⁵ | COOR⁶ | CONR²R³ | COOH |
| 1 | decyl | 0.5 | ethyl | 3.0 | H | decyl | 0.5 | 9 | 16 | 12 | 63 |
| 2 | cetyl | 0.5 | ethyl | 2.75 | H | cetyl | 0.75 | 12 | 10 | 16 | 62 |
| 3 | oleyl | 2.5 | ethyl | 1.0 | Methyl | dodecyl | 0.5 | 54 | 2 | 10 | 34 |
| 4 | cetyl | 1.0 | ethyl | 2.5 | H | hydroxyethyl | 0.5 | 22 | 14 | 11 | 53 |
| 5 | | | ethyl | 3.0 | H | cetyl | 1.0 | | 21 | 22 | 57 |
| 6 | cetyl | 1.5 | ethyl | 1.5 | H | ethyl | 1.0 | 34 | 5 | 20 | 31 |
| Comparative | cetyl | 1.0 | ethyl | 3.00 | | | | 20 | 16 | | 64 |
| Example | | | | | | | | | | | |

### Example 7

O/W emulsion containing polyaspartic acid derivatives

Cetyl polyaspartate from Example 2

| | |
|---|---|
| (25% in water, pH 5.5) | 2.0% |
| Glycerol | 3.0% |
| Preservative | 0.1% |
| Water | 70.0% |
| Glycerol monostearate (Tegin ® M, | |
| Th. Goldschmidt) | 4.5% |
| Tegosoft ® CT (caprylic/capric triglyceride, | |
| Th. Goldschmidt) | 20.00% |

The aqueous phase and the oily substance/glycerol monostearate mixture were intensively mixed at 70° C. using a rotor-stator homogenizer (SG/220V, 2 min). The emulsion (100 ml) was stored for 2 days at 20° C. and 7 d at 45° C. Sensory evaluation of the sample showed, in the case of Example 7, no change of the creamy consistency, and in the case of Comparative Example 2, a drop in viscosity. Water separation in the W/O emulsions was determined after storage for 2 days at 20° C. and after storage for a further 7 days at 45° C.

| | | WATER SEPARA- | WATER SEPARA- |
|---|---|---|---|
| | EMULSIFIER | TION AFTER | TION AFTER |
| | FROM | 2 DAYS/20° C. | 28 DAYS/45° C. |
| EXAMPLE | EXAMPLE | (% BY VOLUME) | (% BY VOLUME) |
| 7 | 2 | <0.1% | <0.1% |
| Comparative | Comparative | <0.1% | 1.0% |
| Example 2 | Example 2 | | |

The results show the increased emulsion stability in the case of the amide-modified polyaspartic esters.

### Example 8

Foaming surfactant preparation containing polyaspartic acid derivatives:

| | |
|---|---|
| (A) | (B) |

| | |
|---|---|
| Formulation | [% by weight] |

| | | |
|---|---|---|
| Product as in Example 1 | | |
| (50% strength in water, pH 5.5) | 0.0% | 1.0% |
| Texapon ® N28 (28% sodium lauryl | 21.4% | 21.4% |
| ether sulfate, Henkel) | | |
| Tego ® betaine E50 (37.5% cocoamido- | 16.0% | 16.0% |
| propylbetaine, Th. Goldschmidt) | | |
| Water ad 100%, pH ad 6.0 | | |

The foaming properties of the surfactant mixture were determined by foaming a dilute surfactant solution. (0.5% by weight of active detergent substance, 8° German hardness, 30° C., Ystral guide-beam mixer, 750 W, 2 min)

| | | WATER | |
|---|---|---|---|
| | FOAM VOLUME | SEPARATION | FOAM DENSITY |
| MIXTURE | [ml] | 10 MIN [ml] | [g/ml] |
| A | 1490 ± 17 | 240 ± 2.0 | 0.208 ± 0.002 |
| B | 1573 ± 10 | 236 ± 2.9 | 0.191 ± 0.003 |

This example demonstrates the beneficial effect of the polyaspartic acid derivatives on the foaming behavior of surfactant systems.

### Example 9

| | |
|---|---|
| Polyaspartic acid derivatives as in Example 1, | 11.0% |
| (50% strength in water, pH 5.5) | |
| Texapon ® N70 (70% sodium lauryl ether sulfate, | 32.0% |
| Henkel) | |
| Tagat ® R40 (PEG-40 ethoxylate of hydrogenated | 5.0% |
| castor oil, Th. Goldschmidt) | |
| Tego ® glucoside 810 (60% caprylic/capric glucoside, | 8.0% |
| Th. Goldschmidt) | |
| Citric acid (20%) | 0.9% |
| NaC1 (25%) | 8.5% |
| Water | 16.6% |
| Tego ® betaine F50 (37.5% cocoamidopropylbetaine, | 18.0% |
| Th. Goldschmidt) | |

### Example 10

O/W based care cream

| | |
|---|---|
| Polyaspartic acid derivative as in Example 2, | 4.0% |
| (50% strength in water, pH 5.5) | |
| Polyaspartic acid derivative as in Example 4, | 1.0% |
| (50% strength in water, pH 5.5) | |
| Tego ® Care 450 (polyglyceryl-3 methylgiucoside | 1.0% |
| distearate, Th. Goldschmidt) | |
| Tegin ® M (glyceryl stearate, Th. Goldscbmidt) | 0.5% |
| Tego ® Akanol 18 (stearyl alcohol, Th. Goldschmidt) | 0.3% |
| Avocado oil | 12.0% |
| Tegosoft ® CT (caprylic/capric triglyceride, | 9.0% |
| Th. Goldschmidt) | |
| Glycerol | 3.0% |
| Water | 69.2% |
| NaOH (10%) ad pH 5.5 | |

### Example 11

W/O Cream

| | |
|---|---|
| Polyaspartic acid derivative as in Example 3 | 3.0% |
| (50% strength in water, pH 5.5) | |
| Abil ® EM90 (cetyl polyether siloxane, | 1.5% |
| Th. Goldscbmidt) | |
| Isolan ® GI 34 (polyglyceryl-4 isostearate, | 0.8% |
| Th. Goldscbmidt) | |
| Avocado oil | 11.0% |
| Tegosoft ® CT (caprylic/capric triglyceride, | 11.0% |
| Th. Goldschmidt) | |
| hydrogenated castor oil | 0.8% |
| Beeswax | 1.0% |
| NaC1 | 0.5% |
| Water | 0.5% |
| Fragrances, preservatives | 70.4% |

While the invention has been particularly shown and described with respect to preferred embodiments thereof, it will be understood by those skilled in the art that the foregoing and other changes in form and details may be made therein without departing from the spirit and scope of the invention.

1. A copolymer derived from polyamino acids, in which at least 75 mol % of the units present consist of structural units of the general formulae (I), (II) or (III) 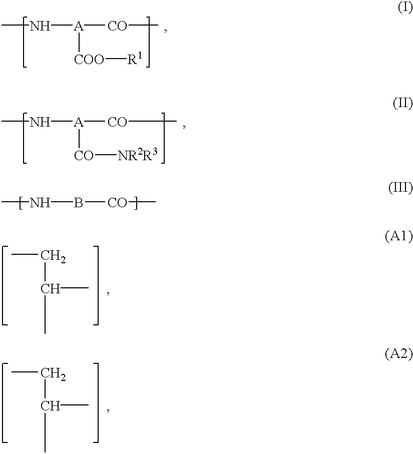
in which the structural elements A are identical or different trifunctional hydrocarbon radicals having 2 carbon atoms of the type (A1) or (A2), where
R¹ may have the meanings of R⁴, R⁵, and R⁶,
R² is identical or different, straight-chain or branched, saturated or unsaturated alkyl, alkenyl or aryl radicals having from 1 to 30 carbon atoms, hydroxyl or aminoalkyl radicals having from 1 to 22 carbon atoms and from 1 to 6 hydroxyl and/or amino groups and/or their acylation products with from 1 to 22 C carboxylic acids or assumes the meaning of R⁵, and
R³ is hydrogen or R²,
R4 is one or more radicals from the group consisting of alkali metals, alkaline earth metals, hydrogen or ammonium, [NR⁷R⁸R⁹R¹⁰]⁺, where
R⁷ to R¹⁰ independently of one another are hydrogen, alkyl or alkenyl having from 1 to 22 carbon atoms or hydroxyalkyl having from 1 to 22 carbon atoms, having from 1 to 6 hydroxyl groups.
R⁵ is identical or different, straight-chain or branched, saturated or unsaturated alkyl or alkenyl radicals R¹¹ having from 6 to 30 carbon atoms or radicals of the structure —Y—R¹¹, where Y is an oligo- or polyoxyalkylene chain having from 1 to 100 oxyalkylene units, and
R⁶ is identical or different, straight-chain or branched, saturated or unsaturated alkyl or alkenyl radicals having from 1 to 5 carbon atoms, and in each case at least one unit of the general formula (I) in which the radical R¹ assumes the meaning of R⁴, and at least one unit of the general formula (II) is present, and the units of the general formula (III) are proteinogenic or nonproteinogenic amino acids and are present in amounts of no more than 20% by weight, based on copolymeric polyaspartic acid derivatives.

2. The copolymer as claimed in claim 1, in which at least one radical R¹ assumes the meaning of R⁵ or R⁶.

3. The copolymer as claimed in claim 1, in which at least one radical R¹ or R² assumes the meaning of R⁵.

4. The copolymer as claimed in claim 1, in which the structural elements II are derived from primary or secondary amines NR²R³H, in which R² is a straight-chain or branched, saturated or unsaturated alkyl, alkenyl or aryl radical having from 1 to 30 carbon atoms, and R³ is hydrogen or methyl.

5. The copolymer as claimed in claim 1, in which the structural elements (II) are derived from aminoalcohols such as ethanolamine, diethanolamine, and/or aminopropanols and from their N-methyl derivatives or their acylation products.

6. The copolymer as claimed in claim 1, which is prepared in the presence of molecular-mass-increasing agents selected from the group consisting of di- or polyhydroxy compounds, di- or polyamino compounds, or aminoalcohols or mixtures thereof, having a linear, branched or cyclic, saturated, unsaturated or aromatic hydrocarbon backbone, optionally oxo- or aza-substituted by O or N atoms in the chain, or their reaction products with maleic anhydride, or is modified therewith following the preparation.

7. A process for the preparation of a copolymer as claimed in claim 1, which comprises the amides or the esters and amides of α, β-unsaturated dicarboxylic acids or their ammonium salts, in particular maleic acid derivatives of the general formulae (V), (VI) and (VII) 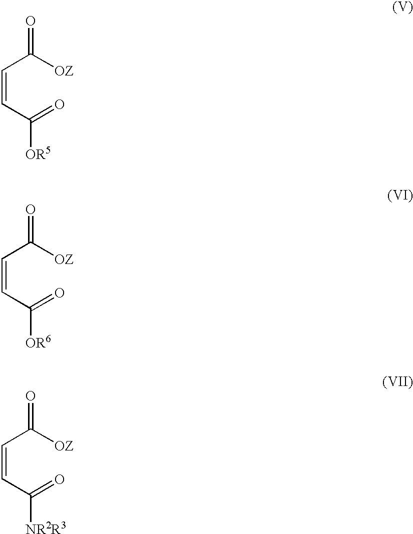
alone or mixed with one another, being reacted with ammonia and converted into the polymer, where
Z is hydrogen or ammonium,
R², R³, R⁵ and R⁶ are the abovementioned radicals, optionally in the presence of up to 20% by weight, based on copolymeric polyaspartic acid derivatives, of proteinogenic or nonproteinogenic amino acids or their derivatives, and, optionally in further steps, producing, by hydrolysis, groups of the structure of the formula (I), where
R¹ has the meaning of R⁴, with the definition of R⁴ stated above.

8. A cosmetic W/O or O/W emulsion comprising copolymeric polyaspartic acid derivatives as claimed in claim 1.

9. The cosmetic emulsion as claimed in claim 8, wherein the nonaqueous fraction comprises from 5 to 99% by weight of oily substances from the group consisting of esters of linear C₆-C₂₀ fatty acids with linear C₆-C₂₀ fatty alcohols, esters of branched C₆-C₁₃ carboxylic acids with linear C₆-C₂₀ fatty alcohols, esters of linear C₆-C₂₀ fatty acids with branched alcohols, esters of linear and/or branched C₆-C₂₀ carboxylic acids with polyhydric alcohols and/or Guerbet alcohols, triglycerides based on C₆-C₁₀ fatty acids, vegetable and animal oils and fats, branched primary alcohols, substituted cyclohexanes, Guerbet carbonates, dialkyl ethers and/or aliphatic or naphthenic hydrocarbons.

10. The cosmetic O/W emulsion as claimed in claim 8, wherein hydrophilic waxes from the group consisting of C₁₂-C₃₀-fatty alcohols, wool wax alcohols, C₁₆-C₂₂-fatty acids, glycerol mono- and diesters and sorbitan mono- and diesters of saturated fatty acids having from 12 to 22 carbon atoms are present.

11. The cosmetic emulsion as claimed in claim 8, wherein one or more coemulsifiers from the group consisting of the addition products of ethylene oxide or ethylene oxide and propylene oxide with C₁₂-C₃₀-fatty alcohols and wool wax alcohols, of ethylene oxide addition products of glycerol mono- and diesters and sorbitan mono- and diesters of saturated and unsaturated fatty acids having from 6 to 22 carbon atoms, of addition products of ethylene oxide and/or propylene oxide with fatty acids having from 12 to 22 carbon atoms and with alkylphenols having from 8 to 15 carbon atoms in the alkyl group, of C₁₂-C₈-fatty acid mono- and diesters of addition products of ethylene oxide with glycerol, of addition products of ethylene oxide with fats and oils, of polyol esters of saturated or unsaturated C₁₂₋₂₂-fatty acids, also branched or hydroxy-substituted, polysiloxane-polyalkyl-polyether copolymers and their derivatives, of anionic surfactants, cationic surfactants, nonionic surfactants and zwitterionic or amphoteric surfactants are present.

12. A surfactant preparation for cleansers and/or cosmetic compositions, comprising polyaspartic acid derivatives as claimed in claim 1, optionally comprising one or more further surfactants from the group of anionic, cationic, nonionic, amphoteric and zwitterionic surfactants and the mixtures thereof, as well as customary auxiliaries and additives.
